(19) United States
(12) Patent Application Publication
(10) Pub. No.: US 2009/0062239 A1
(43) Pub. Date: Mar. 5, 2009

(54) THROMBIN RECEPTOR ANTAGONISTS AS PROPHYLAXIS TO COMPLICATIONS FROM CARDIOPULMONARY SURGERY

(75) Inventors: Enrico P. Veltri, Princeton, NJ (US); John T. Strony, Lebanon, NJ (US); Gail Berman, Westfield, NJ (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(73) Assignee: Schering Corporation

(21) Appl. No.: 12/261,844

(22) Filed: Oct. 30, 2008

Related U.S. Application Data
(63) Continuation of application No. 11/613,450, filed on Dec. 20, 2006, now abandoned.
(60) Provisional application No. 60/753,246, filed on Dec. 22, 2005.

(51) Int. Cl. A61K 31/443; A61P 9/00; A61K 31/444; A61K 31/60; A61K 31/4365

(52) U.S. Cl. 514/161; 514/337; 514/333; 514/301

(57) ABSTRACT

Disclosed herein are methods of preventing, inhibiting, or ameliorating complications associated with cardiopulmonary bypass surgery by the use of a thrombin receptor antagonist compound. Among the thrombin receptor antagonist compounds useful in these methods are those of Formulas I and II, described herein. Examples of such thrombin receptor antagonists include:

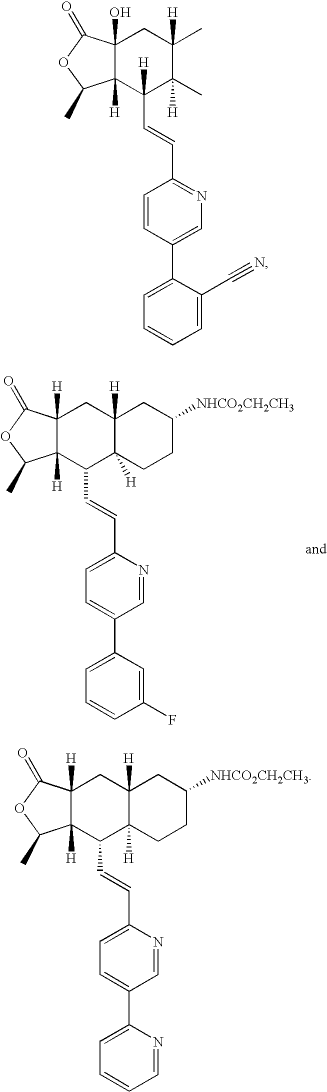

### CROSS-REFERENCE TO RELATED APPLICATIONS

This application claims the benefit of U.S. Provisional Application No. 60/753,246, filed on Dec. 22, 2005, the text of which is herein incorporated by reference.

### BACKGROUND OF THE INVENTION

Cardiopulmonary bypass surgery (“CPB”) is performed about 709,000 times annually in the Unites States, making it one of the most commonly performed significant major operations. Surgeries that utilize CPB include coronary artery bypass graft surgery (“CABG”), cardiac valvular repair and replacement surgery, pericardial and aortic repair surgeries. Any procedure which includes CPB surgery can involve a set of common risks largely associated with the contacting of circulating blood with the surfaces of the bypass equipment. Such contact can result in clot formulation, which can pose a serious threat of stroke to the patient. CABG surgery can pose additional risks to the patient.

CABG surgery is advised for selected groups of patients with significant narrowings and blockages of the heart arteries (coronary artery disease). CABG surgery creates new routes around narrowed and blocked arteries, allowing sufficient blood flow to deliver oxygen and nutrients to the heart muscles.

Coronary artery disease occurs when atherosclerotic plaque (hardening of the arteries) builds up in the wall of the arteries that supply the heart. This plaque is primarily made of cholesterol. The atherosclerotic process causes significant narrowing in one or more coronary arteries. When coronary arteries narrow more than 50 to 70%, the blood supply beyond the plaque becomes inadequate to meet the increased oxygen demand during exercise. The heart muscle in the territory of these arteries becomes starved of oxygen (ischemic). Patients often experience chest pain (angina) when the blood oxygen supply cannot keep up with demand. Up to 25% of patients experience no chest pain at all despite documented lack of adequate blood and oxygen supply. These patients have “silent” angina, and have the same risk of heart attack as those with angina.

When a blood clot (thrombus) forms on top of this plaque, the artery becomes completely blocked causing a heart attack. When arteries are narrowed in excess of 90 to 99%, patients often have accelerated angina or angina at rest (unstable angina). Unstable angina can also occur due to intermittent blockage of an artery by a thrombus which is dissolved by the body's own protective clot dissolving system.

CABG surgery is performed to relieve angina in patients who have failed medical therapy and are not good candidates for balloon angioplasty. CABG surgery is ideal for patients with multiple narrowings in multiple coronary artery branches, such as is often seen in patients with diabetes. CABG surgery has been shown to improve long-term survival in patients with significant narrowing of the left main coronary artery, and in patients with significant narrowing of all three major arteries, especially in those with decreased heart muscle pump function. CABG surgery may improve long-term survival in patients with significant narrowing of two major arteries with one involving the beginning section of the left anterior descending artery.

However, 5-10% of vein grafts become blocked within the first two weeks after CABG surgery due to blood clotting. Blood clots form in the grafts usually because of small arteries beyond the insertion site of the graft causing sluggish blood run off. Another 10% of vein grafts close off between two weeks and one year after CABG surgery. Use of aspirin to thin the blood has been shown to reduce these later closings by 50%. Grafts become narrowed after the first five years as cells stick to the inner lining and multiply, causing formation of scar tissue (intimal fibrosis) and actual atherosclerosis. After ten years, only two-thirds of vein grafts are open, and half of these have at least moderate narrowings.

Conventional CPB surgery elicits a systemic inflammatory response. During CPB, platelets are exposed to nonendothelial surfaces which triggers activation, aggregation, and platelet loss. This contributes to the increased incidence of hemorrhage and its related sequelae during the immediate peri-operative and post-operative periods. This includes the increased need for transfusion of platelets, red blood cells, cryoprecitate, and/or fresh frozen plasma, as well as the need for surgical re-exploration. Bleeding is inevitable during the procedure, and platelet activation (and thus loss) is markedly worsened by the use of an externalized circulation and pump. The risk of bleeding is further increased by clopidogrel use, so many surgeons will delay CABG five to seven days to allow prior clopidogrel to wash out (per US surgical guidelines and US label).

Homologous blood transfusions after CABG are correlated in a dose-related fashion to increased risk for viral and bacterial infections, increased length of stay, antimicrobial use, and mortality through transfusion-related immunomodulation. (Murphy, P. J., Connery, C., Hicks, G. L., Blumberg, N., Homologous blood transfusion as a risk factor for postoperative infection after coronary artery bypass graft operations. J. Thorac. Cardiovasc. Surg., 1992; 104:1092-9; van de Watering, L. M., Hermans, J., Houbiers, J. G., et al., Beneficial effects of leukocyte depletion of transfused blood on postoperative complications in patients undergoing cardiac surgery: a randomized clinical trial. Circulation, 1998; 97: 562-8). Predisposing risk factors for transfusion after CABG include advancing age, lower preoperative red blood cell volume, preoperative aspirin therapy, priority of operation, duration of CPB, recent fibrinolytic therapy, reoperative CABG, and differences in heparin management. (Eagle, Kim A., Guyton, Robert A., et al., American College of Cardiology Foundation and the American Heart Association, Inc., 2004 Guideline Update for Coronary Artery Bypass Graft Surgery). Aprotinin, a serine protease inhibitor with antifibrinolytic activity, significantly decreases postoperative blood loss and transfusion requirements (both units and number of patients) in high-risk, patients undergoing primary CABG, those on aspirin, and in particular the population undergoing reoperative bypass. (Harder, M. P., Eijsman, L., Roozendaal, K. J., van Oeveren, W., Wildevuur, C. R., Aprotinin reduces intraoperative and postoperative blood loss in membrane oxygenator cardiopulmonary bypass. Ann. Thorac. Surg., 1991; 51: 936-41; Cosgrove, D. M., Heric, B., Lytle, B. W., et al., Aprotinin therapy for reoperative myocardial revascularization: a placebo-controlled study. Ann. Thorac. Surg., 1992; 54: 1031-6).

CPB patients, and in particular, CABG patients with acute coronary syndrome are often treated with new and more potent antithrombotic and antiplatelet therapies which may pose heightened risks of CABG-associated complications. Several studies have demonstrated a greater risk for postoperative hemorrhage in patients treated with low-molecular weight heparin (Clark, S. C., Vitale, N., Zacharias, J., Forty, J., Effect of low molecular weight heparin (fragmin) on bleeding after cardiac surgery., Ann. Thorac. Surg. 2000; 69: 762-4); abciximab (Lincoff, A. M., LeNarz, L. A., Despotis, G. J., et al., Abciximab and bleeding during coronary surgery: results from the EPILOG and EPISTENT trials. Improve Long-term Outcome with abciximab GP IIb/IIIa blockade. Evaluation of Platelet IIb/IIIa Inhibition in STENTing. Ann. Thorac. Surg. 2000; 70: 516-26); and clopidogrel (Yusuf, S., Zhao, F., Mehta, S. R., Chrolavicius, S., Tognoni, G., Fox, K. K., for the Clopidogrel in Unstable Angina to Prevent Recurrent Events Trial Investigators. Effects of clopidogrel in addition to aspirin in patients with acute coronary syndromes without ST segment elevation. N. Engl. J. Med., 2001; 345: 494-502).

Thrombin is known to have a variety of activities in different cell types and thrombin receptors are known to be present in such cell types as human platelets, vascular smooth muscle cells, endothelial cells and fibroblasts.

Thrombin receptor antagonists have been identified based on structure-activity studies involving substitutions of amino acids on thrombin receptors. In Bernatowicz et al, J. Med. Chem., vol. 39, pp. 4879-4887 (1996), tetra- and pentapeptides are disclosed as being potent thrombin receptor antagonists, for example N-trans-cinnamoyl-p-fluoroPhe-p-guanidinoPhe-Leu-Arg-NH₂ and N-trans-cinnamoyl-p-fluoroPhe-p-guanidinoPhe-Leu-Arg-Arg-NH₂. Peptide thrombin receptor antagonists are also disclosed in WO 94/03479, published Feb. 17, 1994.

Thrombin receptor antagonists have been suggested in the literature as being potentially useful in treating a variety of cardiovascular diseases or conditions including, for example, thrombosis, vascular restenosis, deep venous thrombosis, lung embolism, cerebral infarction, heart disease, disseminated intravascular coagulation syndrome, hypertension (Suzuki, Shuichi, PCT Int. Appls. WO 0288092 (2002), WO 0285850 (2002) and WO 0285855 (2002)), arrhythmia, inflammation, angina, stroke, atherosclerosis, ischemic conditions (Zhang, Han-cheng, PCT Int. Appl. WO 0100659 (2001), WO 0100657 (2001) and WO 0100656 (2001)).

Substituted thrombin receptor antagonists are disclosed in U.S. Pat. Nos. 6,063,847; 6,326,380; and 6,645,987 and U.S. publication nos. 03/0203927; 04/0216437A1; 04/0152736; and 03/0216437. The use of a small subset of thrombin receptor antagonists to treat a variety of conditions and diseases is disclosed in U.S. publication no. 04/0192753. A bisulfate salt of a particular thrombin receptor antagonist is disclosed in 2004/0176418A1.

### SUMMARY OF THE INVENTION

The present invention is directed to a method of preventing, inhibiting, or ameliorating a condition associated with cardiopulmonary bypass surgery comprising administering an effective amount of at least one thrombin receptor antagonist compound to a subject of said surgery.

In some embodiments, the condition is selected from at least one of the group consisting of: bleeding; thrombotic vascular events such as thrombosis, restenosis; vein graft failure; artery graft failure; atherosclerosis, angina pectoris; myocardial ischemia; acute coronary syndrome myocardial infarction; heart failure; arrhythmia; hypertension; transient ischemic attack; cerebral function impairment; thromboembolic stroke; cerebral ischemia; cerebral infarction; thrombophlebitis; deep vein thrombosis; and, peripheral vascular disease.

In some embodiments, the thrombin receptor antagonist compound is a compound of either Formula I or II, as described infra. In some embodiments, the thrombin receptor antagonist is E-5555. In some embodiments, the thrombin receptor antagonist is selected from at least one of the group of compounds consisting of the following:

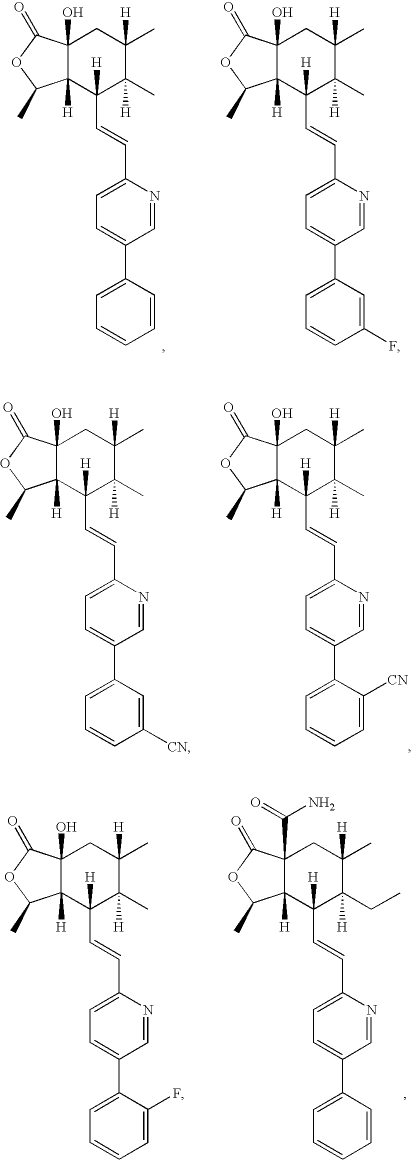

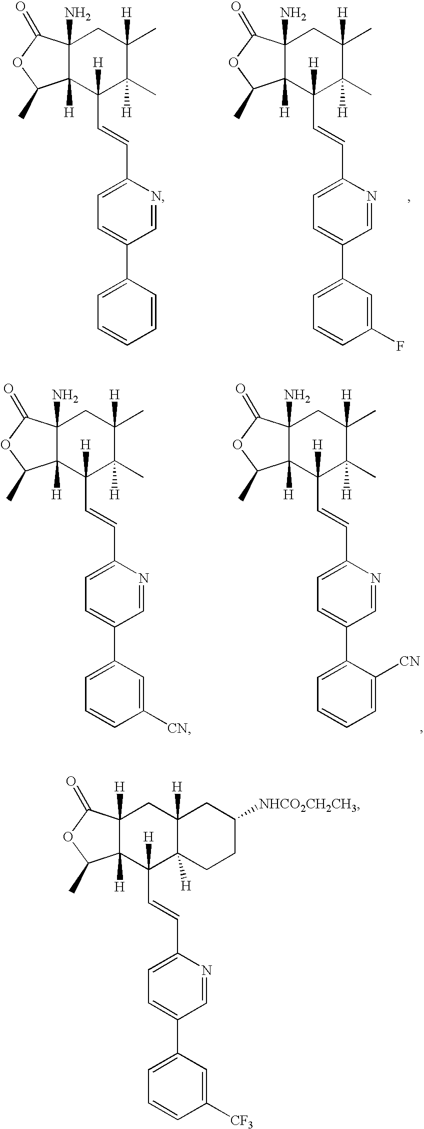

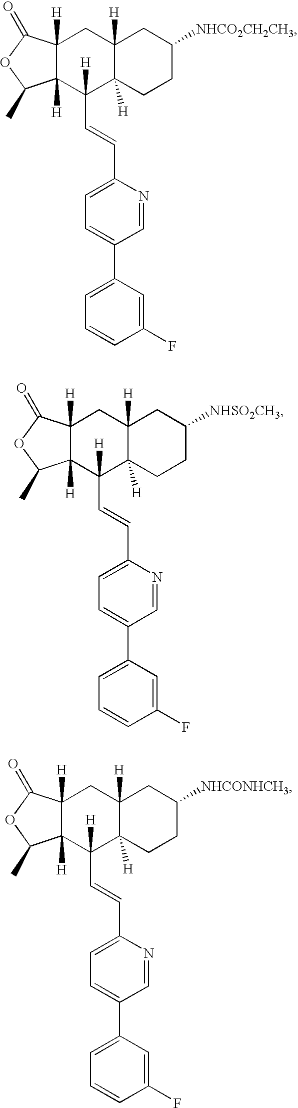

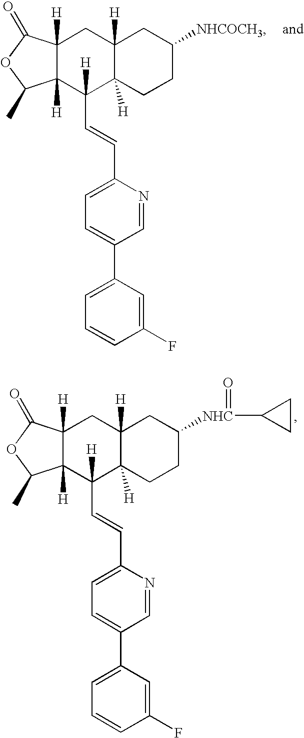

or a pharmaceutically acceptable isomer, salt, solvate or co-crystal form thereof.

In some embodiments, the thrombin receptor antagonist compound is selected from at least one of the group of compounds consisting of the following:

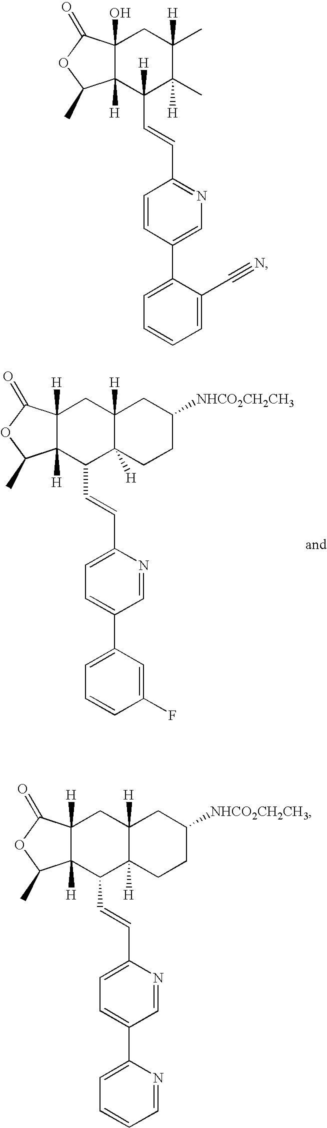

or a pharmaceutically acceptable isomer, salt, solvate or co-crystal form thereof.

In some embodiments, the method further comprises administering at least one cardiovascular agent selected from the group consisting of thromboxane A2 biosynthesis inhibitors; thromboxane antagonists; adenosine diphosphate inhibitors; cyclooxygenase inhibitors; angiotensin antagonists; endothelin antagonists; phosphodiesterase inhibitors; angiotensin converting enzyme inhibitors; neutral endopeptidase inhibitors; anticoagulants; diuretics; platelet aggregation inhibitors; and GP IIb/IIIa antagonists.

In some embodiments, the method further comprises administering at least two of said cardiovascular agents.

In some embodiments, the method further comprises administering at least one cardiovascular agent selected from the group consisting of aspirin, seratrodast, picotamide and ramatroban, clopidogrel, meloxicam, rofecoxib, celecoxib, valsartan, telmisartan, candesartran, irbesartran, losartan, eprosartan, tezosentan, milrinone, enoximone, captopril, enalapril, enaliprilat, spirapril, quinapril, perindopril, ramipril, fosinopril, trandolapril, lisinopril, moexipril, benazapril, candoxatril, ecadotril, ximelagatran, fondaparin, enoxaparin, chlorothiazide, hydrochlorothiazide, ethacrynic acid, furosemide, amiloride, abciximab, eptifibatide, prasugrel and fragmin.

In some embodiments, the method further comprises administering at least two of said cardiovascular agents.

In some embodiments, the thrombin receptor antagonist compound is

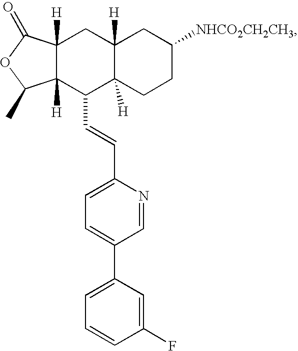

or a pharmaceutically acceptable isomer, salt, solvate or co-crystal form thereof.

In some embodiments, the thrombin receptor antagonist compound is

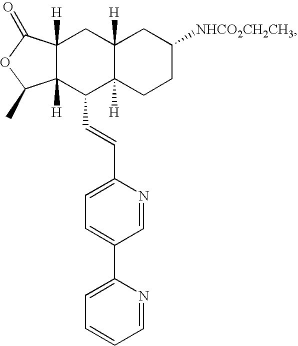

or a pharmaceutically acceptable isomer, salt, solvate or co-crystal form thereof.

In some embodiments, the thrombin receptor antagonist compound is

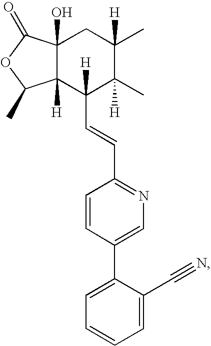

or a pharmaceutically acceptable isomer, salt, solvate or co-crystal form thereof.

In some embodiments, the thrombin receptor antagonist compound is administered according to a dosing regimen comprising administration of a maintenance dose of about 0.5 to about 10 mg. In some embodiments, the dosing regimen further comprises administration of a loading dose of about 10 to about 50 mg. prior to administration of the first maintenance dose.

In some embodiments, the method comprises preventing a condition associated with coronary arterial bypass graft surgery comprising administering an effective amount of a compound of the formula

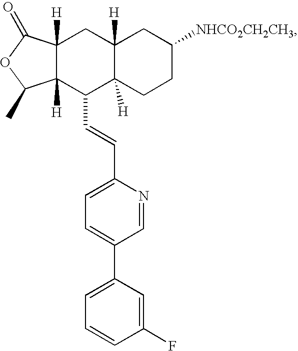

to a subject of said surgery, wherein said condition is at least one of: bleeding; thrombotic vascular events such as thrombosis, restenosis; vein graft failure; artery graft failure; atherosclerosis, angina pectoris; myocardial ischemia; acute coronary syndrome myocardial infarction; heart failure; arrhythmia; hypertension; transient ischemic attack; cerebral function impairment; thromboembolic stroke; cerebral ischemia; cerebral infarction; thrombophlebitis; deep vein thrombosis; and, peripheral vascular disease. In some embodiments, this thrombin receptor antagonist is administered according to a dosing regimen comprising administering a maintenance dose of about 0.5 to about 10 mg. In some embodiments, the dosing regimen further comprises administering a loading dose of about 10 to about 50 mg. prior to administration of the maintenance dose. In some embodiments, the method further comprises administering at least one of aspirin, clopidogrel, prasugrel and fragmin.

### DETAILED DESCRIPTION

It is presently believed by the inventors that the use of the above-described thrombin receptor antagonists will be found to be advantageous in the period directly before, during and/or after the CPB procedure in achieving a number of important goals. Although some of the studies cited below were made with respect to CABG, most of the conclusions will be applicable to any procedure involving CPB.

### Platelet Protection and a Reduced Need for Transfusion

It is presently believed by the inventors that thrombin receptor antagonists will have two potential benefits in the CPB setting with regard to protecting platelets and avoiding or decreasing the need for transfusions. First, it is presently believed by the inventors that their antithrombotic activity will inhibit platelet activation within the pump and directly reduce the need for platelet or blood transfusion. Second, because clopidogrel has a bleeding liability, its use is avoided in settings in which CABG is a possibility, e.g., in the emergency room (“ER”) for a patient presenting with Acute Coronary Syndrome (“ACS”). It is presently believed by the inventors that the thrombin receptor antagonists will have a reduced bleeding liability, thus providing the opportunity for early use in the ER.

### Myocardial Protection During CABG

Myocardial ischemia and myocardial infarction are potential problems during CABG, probably as a result of mini-thrombi. It is presently believed by the inventors that the above-described thrombin receptor antagonists will be useful in the prevention of formation of these mini-thrombi, and thus prevention of myocardial ischemia and/or myocardial infarction.

### Cerebral Protection

Cerebral function may be impaired after CABG, possibly as a consequence of the by-pass pump/circulation. The mechanism of this effect is not certain, but mini-emboli to the cerebrovascular bed have been hypothesized. It is presently believed by the inventors that the above-described thrombin receptor antagonists will be useful in preventing or reducing this effect by avoiding formation of the mini-emboli.

### Prevention of Early Coronary Graft Failure

Surgeons now use two different types of coronary conduit—vein grafts and arterial grafts. They have different natural histories, with arterial grafts having a superior survival rate. Vein grafts may fail from thrombosis or from myo-intimal hyperplasia. Results of studies in which aspirin and/or clopidogrel were administered suggest that early vein graft survival is improved by anti-platelet therapy, but at a cost of increased bleeding. It is presently believed by the inventors that the above-described thrombin receptor antagonists will impart graft patency and survival, but without the observed bleeding liability.

### Prevention of Subsequent Thrombotic Vascular Events

Patients undergoing CABG have usually identified themselves as having at least coronary atherosclerosis, and probably diffuse disease. Thus, they are at an increased risk of subsequent thrombotic vascular events, such as thrombosis, restenosis, vein graft failure atherosclerosis, angina pectoris, myocardial ischemia, acute coronary syndrome, myocardial infarction, heart failure, arrhythmia, hypertension, cerebral functional impairment, transient ischemic attack, cerebral ischemia, cerebral infarction, thromboembolic stroke, venous thromboembolism, deep vein thrombosis, peripheral vascular disease, and other cardiovascular diseases. The risk of the occurrence of such subsequent thrombotic events would likely be reduced by the below-described thrombin receptor antagonists.

Thus, CABG involves a cluster of medical risks that may be managed by platelet inhibition. However, the degree of surgical insult necessarily encompassed by the CABG procedure makes bleeding a major risk factor to be considered in the selection of any concomitant therapy. The reduced bleeding liability that the above-described thrombin receptor antagonists are believed to exhibit relative to other platelet inhibiting agents makes them particularly attractive candidates for such therapy.

It is presently believed by the inventors that thrombin receptor antagonists will impart similar benefits in preventing complications associated with procedures other than CABG in which blood is exposed to an artificial surface that promotes thrombosis. Such procedures include any use of cardiopulmonary bypass, as well as implantations of prosthetic valves, indwelling catheters and stents.

As used above, and throughout the specification, the following terms, unless otherwise indicated, shall be understood to have the following meanings:

“Subject” includes both mammals and non-mammalian animals.

“Mammal” includes humans and other mammalian animals.

“Polymorph” means a crystalline form of a substance that is distinct from another crystalline form but that shares the same chemical formula. Polymorphous forms of the compounds of Formula I or II, whether crystalline or amorphous, also are contemplated as being part of this invention.

It should also be noted that any formula, compound, moiety or chemical illustration with unsatisfied valences in the present specification and/or claims herein is assumed to have sufficient hydrogen atom(s) to satisfy the valences.

“Effective amount” or “therapeutically effective amount” is meant to describe an amount of compound or a composition of the present invention effective in antagonism of a thrombin receptor and thus producing the desired therapeutic, ameliorative, inhibitory or preventative effect.

“TRA” is an abbreviation for “thrombin receptor antagonist.”

### TRA Compounds

A variety of families of compounds have been shown to display activity as thrombin receptor antagonists. The compounds of Formula I as disclosed in U.S. Pat. No. 6,645,987 have displayed such activity:

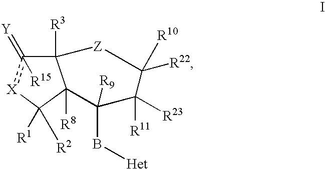

wherein the variables are as defined in U.S. Pat. No. 6,645,987, which is incorporated herein by reference.

As disclosed in U.S. publication no. 2004/0152736, a subset of particularly preferred compounds of Formula I is as follows:

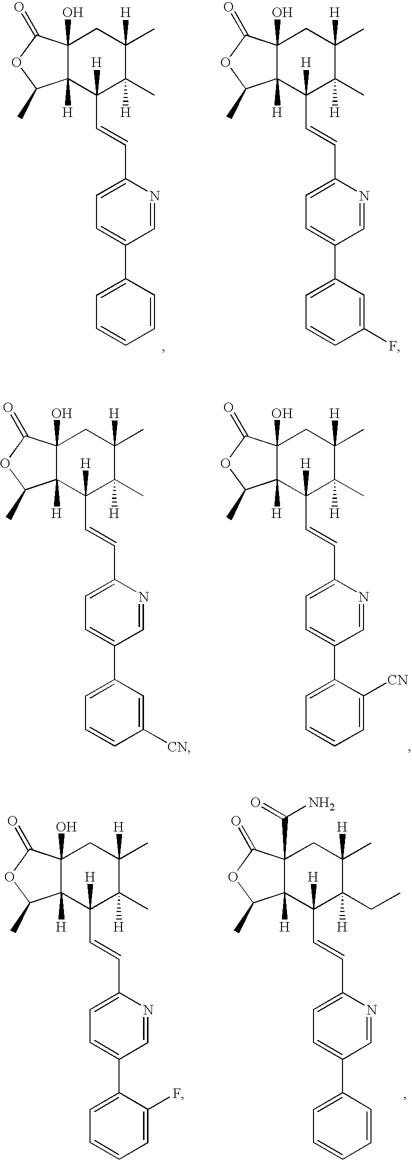

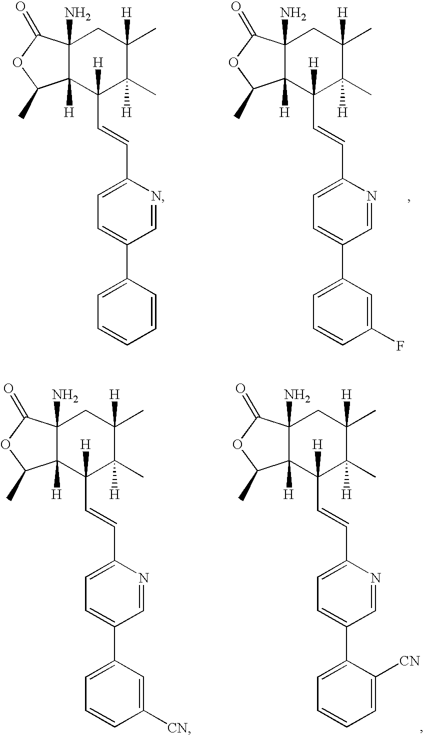

and the pharmaceutically acceptable isomers, salts, solvates and polymorphs thereof.

Further examples of active thrombin receptor antagonists are the compounds of Formula II, and pharmaceutically acceptable salts thereof, as disclosed in U.S. publication no. 2003/0216437:

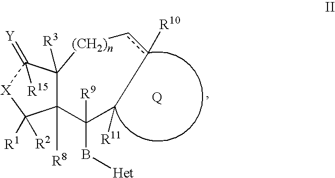

wherein the variables are as defined in U.S. publication no. 2003/0216437, which is incorporated herein by reference.

A particularly active and selective subset of thrombin receptor antagonists of Formula II is as follows:

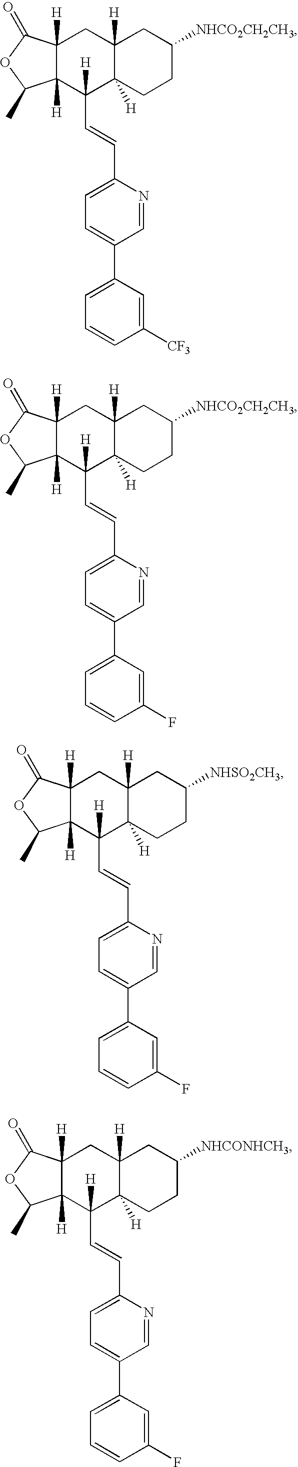

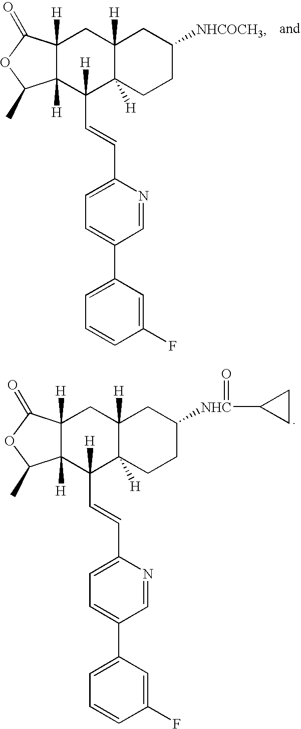

Among the more therapeutically promising thrombin receptor antagonist compounds of Formulas I and II are the following:

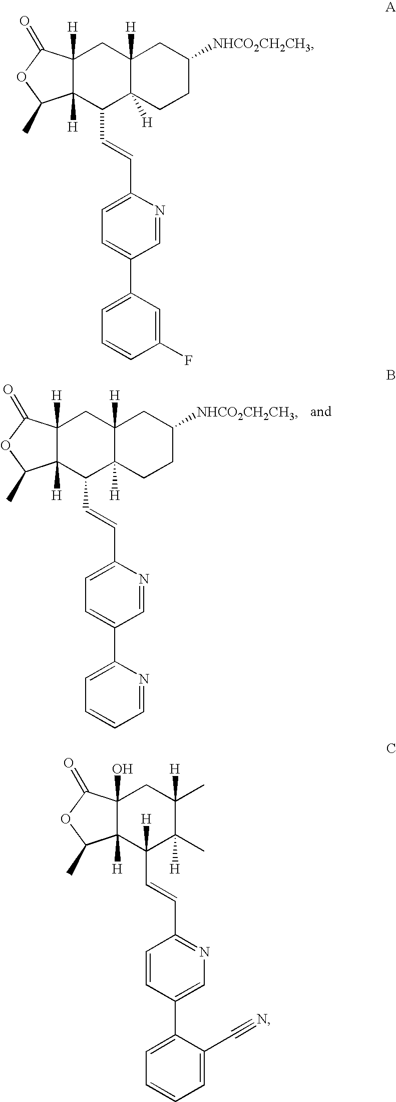

and the pharmaceutically acceptable isomers, salts, solvates and co-crystal forms thereof.

The bisulfate salt of Compound A is currently in development as a thrombin receptor antagonist by Schering-Plough Corp. Its synthesis is disclosed in U.S. publication no. 03/0216437, which publication also discloses Compound C. Compound B is disclosed in U.S. Pat. No. 6,645,987.

Other compounds for use in the formulations of the present invention are disclosed in any of U.S. Pat. Nos. 6,063,847, 6,326,380, U.S. patent Publications U.S. 03/0203927, U.S. 03/0216437, US 04/0192753, and U.S. 04/0176418, the compound-related disclosures of which are all incorporated by reference in their entirety.

The above-described thrombin receptor antagonists are believed to exhibit excellent anti-platelet activity. In addition, they are believed to display a reduced bleeding liability relative to other platelet inhibiting agents, making them particularly attractive candidates as anti-platelet therapies in high bleeding risk scenarios. CPB presents precisely these requirements.

Any other agent that functions as a thrombin receptor antagonist is also within the scope of the present invention. For example, Eisai is currently developing an oral PAR-1 (protease activated receptor) antagonist, designated as E-5555, the structure of which is as follows:

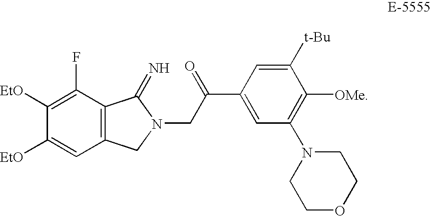

Furthermore, a series of indazole peptidomimetics is reported as displaying activity as thrombin receptor antagonists in U.S. Pat. No. 7,049,297, which is incorporated herein by reference in its entirety. All of these compounds, as well as any other compounds active as thrombin receptor antagonists, are within the scope of this invention.

Some TRA compounds useful in the invention have at least one asymmetrical carbon atom and therefore all isomers, including enantiomers, stereoisomers, rotamers, tautomers and racemates of TRA compounds are contemplated as being part of this invention. The invention includes d and I isomers in both pure form and in admixture, including racemic mixtures. Isomers can be prepared using conventional techniques, either by reacting optically pure or optically enriched starting materials or by separating isomers of a TRA compound. Isomers may also include geometric isomers, e.g., when a double bond is present. Those skilled in the art will appreciate that for some TRA compounds one isomer will show greater pharmacological activity than other isomers.

Typical preferred compounds of Formulas I and II have the following stereochemistries:

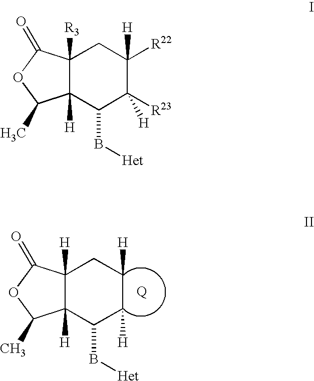

with compounds having these absolute stereochemistries being more preferred.

TRA compounds useful in the invention with a basic group can form pharmaceutically acceptable salts with organic and inorganic acids. Examples of suitable acids for salt formation are hydrochloric, sulfuric, phosphoric, acetic, citric, oxalic, malonic, salicylic, malic, fumaric, succinic, ascorbic, maleic, methanesulfonic and other mineral and carboxylic acids well known to those in the art. Preferred embodiments include bisulfate salts. The salt is prepared by contacting the free base form with a sufficient amount of the desired acid to produce a salt. The free base form may be regenerated by treating the salt with a suitable dilute aqueous base solution such as dilute aqueous sodium bicarbonate. The free base form differs from its respective salt form somewhat in certain physical properties, such as solubility in polar solvents, but the salt is otherwise equivalent to its. respective free base forms for purposes of the invention. TRA compounds useful in the invention can also form pharmaceutically acceptable solvates, including hydrates.

Certain TRA compounds useful in the invention are acidic (e.g., those compounds which possess a carboxyl group). These compounds form pharmaceutically acceptable salts with inorganic and organic bases. Examples of such salts are the sodium, potassium, calcium, aluminum, lithium, gold and silver salts. Also included are salts formed with pharmaceutically acceptable amines such as ammonia, alkyl amines, hydroxyalkylamines, N-methylglucamine and the like.

Prodrugs and solvates of the TRA compounds useful in the invention are also contemplated herein. The term “prodrug”, as employed herein, denotes a compound that is a drug precursor which, upon administration to a subject, undergoes chemical conversion by metabolic or chemical processes to yield a compound of Formula I or II or a salt and/or solvate thereof (e.g., a prodrug on being brought to the physiological pH or through enzyme action is converted to the desired drug form). A discussion of prodrugs is provided in T. Higuchi and V. Stella, Pro-drugs as Novel Delivery Systems (1987) Volume 14 of the A.C.S. Symposium Series, and in Bioreversible Carriers in Drug Design, (1987) Edward B. Roche, ed., American Pharmaceutical Association and Pergamon Press, both of which are incorporated herein by reference thereto.

As used herein, the terms “thrombin receptor antagonist compound” and “TRA compound” are understood to mean any compound displaying activity as a thrombin receptor antagonist, as well as the salts, solvates and hydrates thereof whose preparation would be within the skill of the art. The compounds of Formulas I and II, as well as those disclosed in the references cited herein are non-limiting examples of TRA compounds.

“Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

“Co-crystal” means a crystalline structure simultaneously comprising pharmaceutically active molecules and inert molecules. Co-crystals may be formed by combining a weak base with a weak acid selected to match hydrogen bond donors with acceptors. The pKa difference of conjugate pairs may be inconsistent with salt formation in water. The co-crystallizing agents used to form co-crystals are usually bifunctional acids such as fumaric acid, succinic acid, malic acid, and tartaric acid. Co-crystals are discussed in J. F. Remenar et. al., “Crystal Engineering of Novel Co-crystals of a Triazole Drug with 1,4-Dicarboxylic Acids”, Journal of the American Chemical Society, 2003, vol. 125, pp. 8456-8457.

TRA compounds useful in the invention with a carboxylic acid group can form pharmaceutically acceptable esters with an alcohol. Examples of suitable alcohols include methanol and ethanol.

Compounds of Formulas I and II are prepared by processes described with synthetic schemes and preparative examples disclosed in U.S. Pat. No. 6,645,987 and application Ser. No. 10/412,982, respectively, which schemes and examples are incorporated by reference herein.

### Formulations and Dosing

Prevention of conditions associated with CPB can be effected by administration of a thrombin receptor antagonist to a subject of said surgery. The term “subject” as used herein is understood to mean a mammal, including a human. The term “subject of said surgery” as used herein is understood to mean a mammal for which CPB is planned or which has undergone CPB. Thus, the thrombin receptor antagonist may be administered before, during or after the CPB procedure, depending on such factors as the pharmacokinetic characteristics of the formulation administered and the particular risks faced by the patient. For example, patients at particular risk of bleeding during surgery might be dosed only after surgery, while those at heightened risk of stroke might be dosed before surgery. Formulations resulting in slower bioabsorption might be administered earlier than those with faster bioabsorption rates.

For preparing pharmaceutical compositions from the compounds described by this invention, inert, pharmaceutically acceptable carriers can be either solid or liquid. Solid form preparations include powders, tablets, dispersible granules, capsules, cachets and suppositories. The powders and tablets may be comprised of from about 5 to about 95 percent active ingredient. Suitable solid carriers are known in the art, e.g., magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), The Science and Practice of Pharmacy, 20th Edition, Lippincott Williams & Wilkins, Baltimore, Md., (2000).

Liquid form preparations include solutions, suspensions and emulsions. As an example may be mentioned water or water-propylene glycol solutions for parenteral injection or addition of sweeteners and opacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier, such as an inert compressed gas, e.g., nitrogen.

Also included are solid form preparations which are intended to be converted, shortly before use, to liquid form preparations for either oral or parenteral administration. Such liquid forms include solutions, suspensions and emulsions.

The TRA compounds useful in the invention may also be deliverable transdermally. The transdermal compositions can take the form of creams, lotions, aerosols and/or emulsions and can be included in a transdermal patch of the matrix or reservoir type as are conventional in the art for this purpose.

Preferably the compound is administered orally in a solid dosage form. Orally dissolving formulations of thrombin receptor antagonists are disclosed in U.S. provisional application No. 60/689,207, which is herein incorporated in its entirety by reference.

Preferably, the pharmaceutical preparation is in a unit dosage form. In such form, the preparation is subdivided into suitably sized unit doses containing appropriate quantities of the active component, e.g., an effective amount to achieve the desired purpose.

The dosing regimen for the above-described thrombin receptor antagonists may comprise administration of a loading dose followed by a series of maintenance doses. The term “loading dose” as used herein will be understood to mean a single dose administered prior to the first maintenance dose and intended to rapidly raise the blood concentration level of the TRA compound toward a therapeutically effective level. The term “maintenance dose” will be understood to mean a dose that is serially administered (i.e., at least twice), and which is intended to either slowly raise blood concentration levels of the TRA compound to a therapeutically effective level, or to maintain such a therapeutically effective level. The maintenance may be administered once per day, once over a period of days (e.g., up to 30 days), or more than once per day (e.g., up to 4 times per day).

The loading doses of the present invention preferably contain a thrombin receptor antagonist described above in an amount of about 10 mg to about 50 mg. Doses of 10, 20 and 40 mg are candidates for development of the loading dose. A 40 mg loading dose is planned for administration in a phase III clinical trial directed to acute coronary syndrome. The inventors consider maintenance doses of about 0.5 mg to about 10 mg to be preferred. Doses of 1, 2.5 and 5 mg are candidates for development of the maintenance dose. A 2.5 mg maintenance dose is planned for once per day administration in the above-referenced phase III clinical trial.

In order to achieve rapid onset of action, the loading dose may be in the form of a rapidly disintegrating oral dosage form. Examples of such dosage forms include wet granulation formulations, lyophilized wafers, and effervescent tablets or wafers.

In some circumstances, it may be preferable to forego the loading dose, and to administer only maintenance doses. This may be the case where the surgery is planned sufficiently in the future to achieve adequate blood concentration levels of the thrombin receptor antagonist without the need for a loading dose.

Further embodiments of the invention encompass the administration of a TRA compound along with at least one additional therapeutically effective agent. Therapeutically effective agents that can be used in combination with the compounds of this invention include drugs that are known and used in the treatment of inflammation, rheumatism, asthma, glomerulonephritis, osteoporosis, neuropathy and/or malignant tumors, angiogenesis related disorders, cancer, disorders of the liver, kidney and lung, melanoma, renal cell carcinoma, renal disease, acute renal failure, chronic renal failure, renal vascular homeostasis, glomerulonephritis, chronic airways disease, bladder inflammation, neurodegenerative and/or neurotoxic diseases, conditions, or injuries, radiation fibrosis, endothelial dysfunction, periodontal diseases and wounds. Further examples of therapeutically effective agents which may be administered in combination with the TRA compound include resistance factors for tumor cells towards chemotherapy and proliferation inhibitors of smooth muscle cells, endothelial cells, fibroblasts, kidney cells, osteosarcoma cells, muscle cells, cancer cells and/or glial cells. The therapeutically effective agents may be cardiovascular agents.

Cardiovascular agents that can be used in combination with the novel compounds of this invention include drugs that have anti-thrombotic, anti-platelet aggregation, antiatherosclerotic, antirestenotic and/or anti-coagulant activity. Such drugs are useful in treating thrombosis-related diseases including thrombosis, atherosclerosis, restenosis, hypertension, angina pectoris, arrhythmia, heart failure, myocardial infarction, glomerulonephritis, thrombotic and thromboembolic stroke, peripheral vascular diseases, other cardiovascular diseases, cerebral ischemia, inflammatory disorders and cancer, as well as other disorders in which thrombin and its receptor play a pathological role. Suitable cardiovascular agents are selected from the group consisting of thromboxane A2 biosynthesis inhibitors such as aspirin; thromboxane antagonists such as seratrodast, picotamide and ramatroban; adenosine diphosphate (ADP) inhibitors such as clopidogrel and prasugrel; cyclooxygenase inhibitors such as aspirin, meloxicam, rofecoxib and celecoxib; angiotensin antagonists such as valsartan, telmisartan, candesartran, irbesartran, losartan and eprosartan; endothelin antagonists such as tezosentan; phosphodiesterase inhibitors such as milrinone and enoximone; angiotensin converting enzyme (ACE) inhibitors such as captopril, enalapril, enaliprilat, spirapril, quinapril, perindopril, ramipril, fosinopril, trandolapril, lisinopril, moexipril and benazapril; neutral endopeptidase inhibitors such as candoxatril and ecadotril; anticoagulants such as unfractionated heparin, ximelagatran, fondaparin and enoxaparin; diuretics such as chlorothiazide, hydrochlorothiazide, ethacrynic acid, furosemide and amiloride; platelet aggregation inhibitors such as abciximab and eptifibatide; and GP IIb/IIIa antagonists.

Preferred types of drugs for use in combination with the novel compounds of this invention are thromboxane A2 biosynthesis inhibitors, cyclooxygenase inhibitors and ADP antagonists. Especially preferred for use in the combinations are aspirin, clopidogrel bisulfate, prasugrel and fragmin.

Further embodiments of the invention encompass the administration of TRA compound along with more than one additional therapeutically effective agent. In these embodiments, the additional therapeutically effective agent may or may not be commonly used in the treatment of the same condition. For example, a TRA compound may be administered along with two cardiovascular agents. Alternatively, a TRA compound may be administered along with a cardiovascular agent and a therapeutically effective agent useful in the treatment of inflammation.

When the invention comprises a combination of a TRA compound and one or more other therapeutically effective agents, the two or more active components may be each contained within a distinct dosage form and co-administered simultaneously or sequentially, or alternatively, all contained within a single pharmaceutical composition. In the former case, the components of the combination can be administered individually or together in any conventional dosage form such as capsule, tablet, powder, cachet, suspension, solution, suppository, nasal spray, etc. The dosage of the other therapeutically active agent(s) can be determined from published material, and may range from 1 to 1000 mg per dose.

In this specification, the term “at least one TRA compound” means that one to three different TRA compounds may be used in a pharmaceutical composition or method of treatment. Preferably one TRA compound is used. Similarly, the term “one or more additional cardiovascular agents” means that one to three additional drugs may be administered in combination with a TRA compound; preferably, one additional compound is administered in combination with a TRA compound. The additional cardiovascular agents can be administered sequentially or simultaneously with reference to the TRA compound.

While the present invention has been described in conjunction with the specific embodiments set forth above, many alternatives, modifications and variations thereof will be apparent to those of ordinary skill in the art. All such alternatives, modifications, and variations are intended to fall within the spirit and scope of the present invention.

We claim:

1. A method of preventing, inhibiting, or ameliorating a condition associated with cardiopulmonary bypass surgery comprising administering an effective amount of at least one thrombin receptor antagonist compound to a subject of said surgery.

2. The method of claim 1 wherein said condition is selected from the group consisting of: bleeding; thrombotic vascular events such as thrombosis, restenosis; vein graft failure; artery graft failure; atherosclerosis, angina pectoris; myocardial ischemia; acute coronary syndrome myocardial infarction; heart failure; arrhythmia; hypertension; transient ischemic attack; cerebral function impairment; thromboembolic stroke; cerebral ischemia; cerebral infarction; thrombophlebitis; deep vein thrombosis; and, peripheral vascular disease.

3. The method of claim 1 wherein said thrombin receptor antagonist compound is selected from the group of compounds consisting of the following: 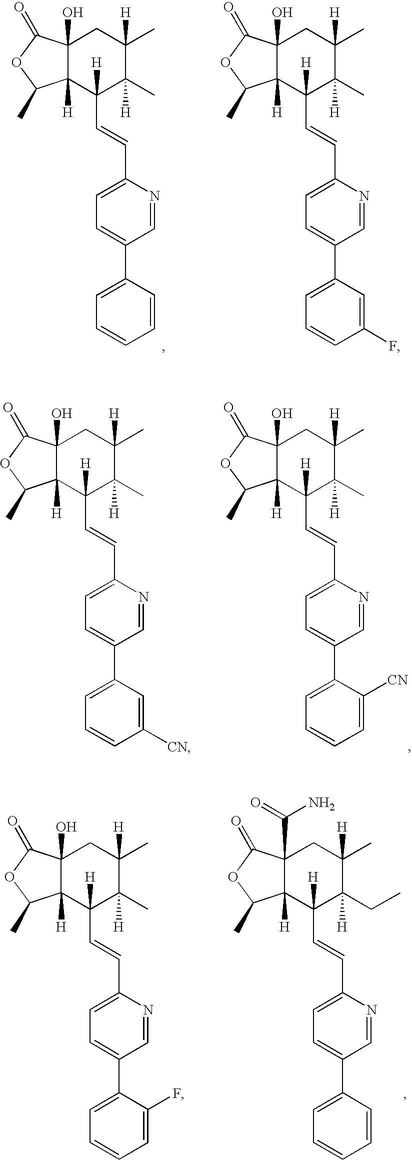 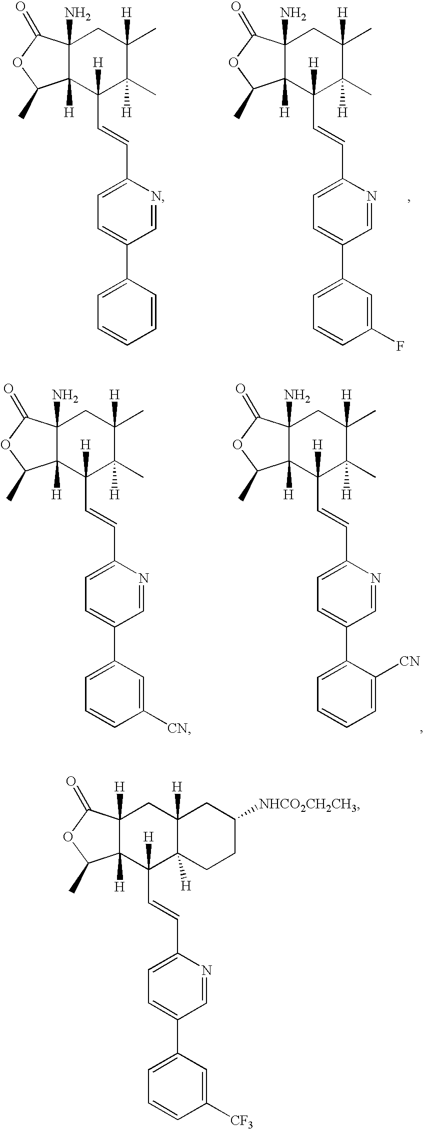 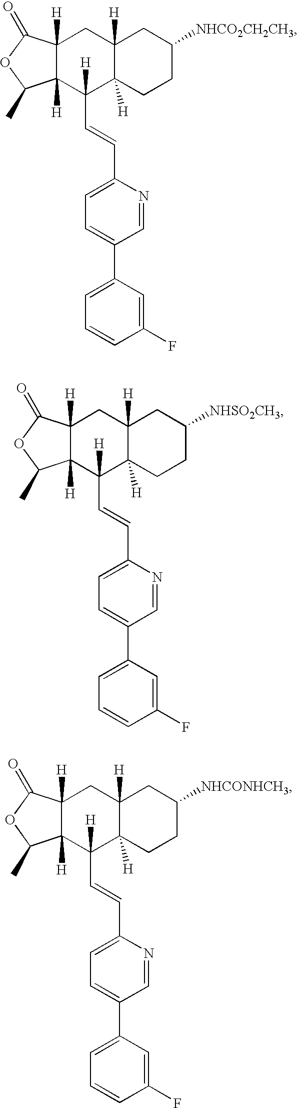 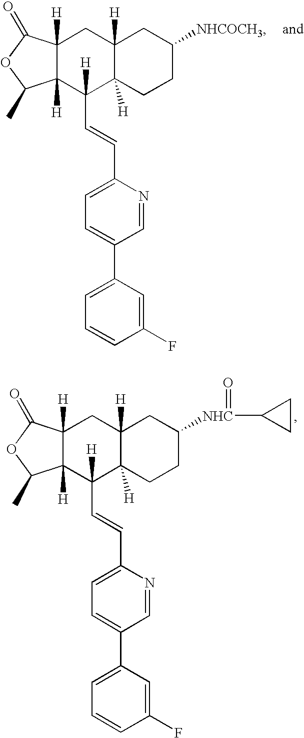
or a pharmaceutically acceptable isomer or salt thereof.

4. The method of claim 2 wherein said thrombin receptor antagonist compound is selected from the group of compounds consisting of the following: 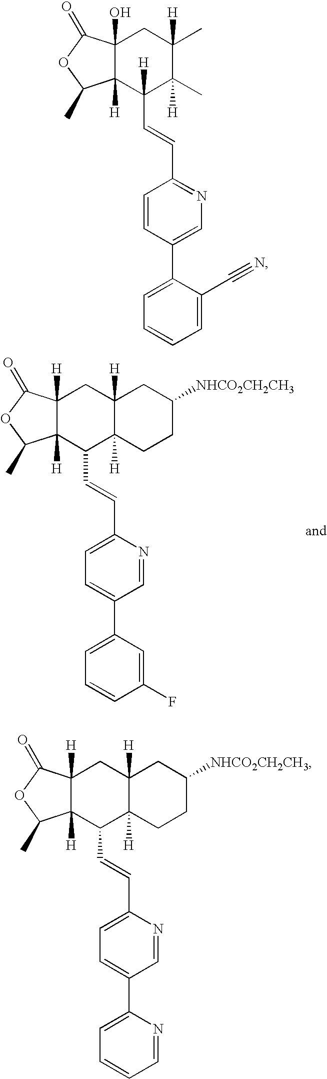
or a pharmaceutically acceptable isomer or salt thereof.

5. The method of claim 1 further comprising administering at least one cardiovascular agent selected from the group consisting of thromboxane A2 biosynthesis inhibitors; thromboxane antagonists; adenosine diphosphate inhibitors; cyclooxygenase inhibitors; angiotensin antagonists; endothelin antagonists; phosphodiesterase inhibitors; angiotensin converting enzyme inhibitors; neutral endopeptidase inhibitors; anticoagulants; diuretics; platelet aggregation inhibitors; and GP IIb/IIIa antagonists.

6. The method of claim 5 further comprising administering at least two of said cardiovascular agents.

7. The method of claim 1 further comprising administering at least one cardiovascular agent selected from the group consisting of aspirin, seratrodast, picotamide and ramatroban, clopidogrel, meloxicam, rofecoxib, celecoxib, valsartan, telmisartan, candesartran, irbesartran, losartan, eprosartan, tezosentan, milrinone, enoximone, captopril, enalapril, enaliprilat, spirapril, quinapril, perindopril, ramipril, fosinopril, trandolapril, lisinopril, moexipril, benazapril, candoxatril, ecadotril, unfractionated heparin, ximelagatran, fondaparin, enoxaparin, chlorothiazide, hydrochlorothiazide, ethacrynic acid, furosemide, amiloride, abciximab, eptifibatide, prasugrel and fragmin.

8. The method of claim 7 further comprising administering at least two of said cardiovascular agents.

9. The method of claim 1 wherein said thrombin receptor antagonist compound is 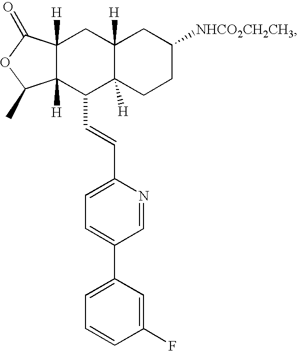
or a pharmaceutically acceptable isomer or salt thereof.

10. The method of claim 1 wherein said thrombin receptor antagonist compound is 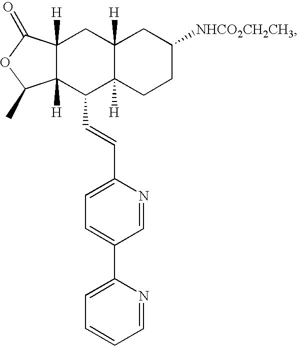
or a pharmaceutically acceptable isomer or salt thereof.

11. The method of claim 1 wherein said thrombin receptor antagonist compound is 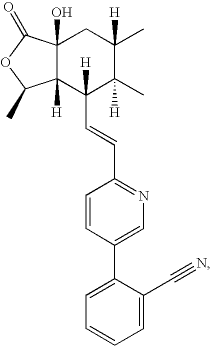
or a pharmaceutically acceptable isomer or salt thereof.

12. The method of claim 1 wherein the thrombin receptor antagonist compound is administered according to a dosing regimen comprising administration of a maintenance dose of about 0.5 to about 10 mg.

13. The method of claim 12 wherein said dosing regimen further comprises administration of a loading dose of about 10 to about 50 mg prior to administration of the first maintenance dose.

14. The method of claim 1, wherein said cardiopulmonary bypass surgery is coronary artery bypass graft surgery.

15. The method of claim 1, wherein said thrombin receptor antagonist compound is E-5555 or a pharmaceutically acceptable isomer or salt thereof.

16. A method of preventing, inhibiting, or ameliorating a condition associated with cardiopulmonary bypass surgery comprising administering an effective amount of a compound of the formula 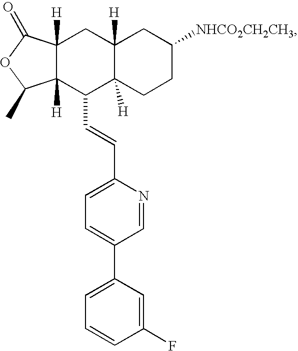
or a pharmaceutically acceptable isomer or salt thereof, to a subject of said surgery, wherein said condition is at least one of: bleeding; thrombotic vascular events such as thrombosis, restenosis; vein graft failure; artery graft failure; atherosclerosis, angina pectoris; myocardial ischemia; acute coronary syndrome myocardial infarction; heart failure; arrhythmia; hypertension; transient ischemic attack; cerebral function impairment; thromboembolic stroke; cerebral ischemia; cerebral infarction; thrombophlebitis; deep vein thrombosis; and, peripheral vascular disease.

17. The method of claim 16 wherein the thrombin receptor antagonist compound is administered according to a dosing regimen comprising administration of a maintenance dose of about 0.5 to about 10 mg.

18. The method of claim 17 wherein the dosing regimen further comprises administration of a loading dose of about 10 to about 50 mg. prior to administration of the first maintenance dose.

19. The method of claim 16 further comprising administering at least one of aspirin, clopidogrel, prasugrel and fragmin.

20. The method of claim 16, wherein said cardiopulmonary bypass surgery is coronary artery bypass graft surgery.
